(19) United States
(12) Patent Application Publication
(10) Pub. No.: US 2012/0310232 A1
(43) Pub. Date: Dec. 6, 2012

(54) SYSTEM AND METHOD FOR TREATING A TISSUE USING MULTIPLE ENERGY TYPES

(76) Inventor: Danny EREZ, Kfar Vitkin (IL)

(21) Appl. No.: 13/489,996

(22) Filed: Jun. 6, 2012

Related U.S. Application Data
(60) Provisional application No. 61/493,550, filed on Jun. 6, 2011.

(51) Int. Cl. A61B 18/18

(52) U.S. Cl. 606/33

(57) ABSTRACT

System and method for treating a tissue using multiple energy types are provided. A system may include at least two flat transducers configured to produce sound waves at surface and inner layer of a skin A plurality of radio frequency (RF) electrodes may be configured to emit a plurality of RF signals. A control unit is to control the transducers such that an interference of the sound waves sustains a predefined level of energy at a specific tissue below the surface of the skin Other embodiments are described and claimed.

### CROSS REFERENCE TO RELATED APPLICATIONS

This application claims the benefit of U.S. Provisional Application No. 61/493,550, filed on Jun. 6, 2011, which is incorporated herein in its entirety.

### FIELD OF THE INVENTION

This application relates to skin treatment, and particularly to treatment of skin using a combination of non-focused ultrasound and radio frequency energies.

### BACKGROUND OF THE INVENTION

A variety of treatment methods are available to improve skin appearance including cosmetic products, devices such as lasers and radio-frequency (RF) devices and combined treatments involving radio frequency (RF) and vacuum. RF may be applied as part of non-ablative deep dermal heating treatment for skin tightening, body shaping and other therapies. Certain applications may apply RF energy to a relatively large area of a soft tissue. In some systems or methods, several small areas of tissue may be treated through the concurrent application of RF through a group of small electrodes that may be brought into contact with the tissue. Some products offer treatments using a combination of energy types in a synchronized manner. For example, intense pulsed light (IPL) energy may be combined with RF energy.

### SUMMARY OF THE INVENTION

Embodiments of the invention may enable a system and method for treating a tissue using multiple energy types. A system may include at least two flat transducers configured to produce sound waves at surface and inner layer of the skin, a plurality of radio frequency (RF) electrodes configured to emit a plurality of RF signals and a control unit. The control unit may cause the transducers to generate sound waves such that an interference of the sound waves sustains a predefined level of energy at a specific tissue below the surface of the skin The control unit may cause the transducers to produce sound waves according to a first and second parameters in order to sustain a respective first and second predefined levels of energies at a respective first and second layers of the skin

Two or more transducers may be respectively positioned to produce a constructive interference of sound waves at a specific layer of the skin Two or more of the RF electrodes may be used and/or controlled to produce a bipolar RF signal according to a predefined sequence. A subset of the RF electrodes may be used and/or controlled to produce a multipolar RF signal. A selected RF electrode and a first subset of RF electrodes may be used and/or controlled to induce a first electro-magnetic field in a first layer of the skin and, subsequently, the selected RF electrode and a second subset of RF electrodes may be used and/or controlled to induce a second electro-magnetic field in a second layer of the skin

A first and second RF electrodes may be selected for emission of energy based on a distance between the first and second RF electrodes. Sound waves may be generated such that cells included in a specific layer of the skin vibrate at a resonance frequency of the cells. A negative pressure outlet may apply a negative pressure to an area of the skin and a receptacle may collect materials released from the skin and translated by the negative pressure. A system may include a cooling unit to cool an area of the skin.

A system may include a sensor the control unit may control the RF electrodes and the transducers according to an input from the sensor. The control unit may control the RF electrodes and the transducers according to input from a user. A plurality of RF electrodes may be arranged substantially around an area of the skin to be treated.

### BRIEF DESCRIPTION OF THE DRAWINGS

The subject matter regarded as the invention is particularly pointed out and distinctly claimed in the concluding portion of the specification. The invention, however, both as to organization and method of operation, together with features and advantages thereof, may best be understood by reference to the following detailed description when read with the accompanied drawings, in which like reference numerals indicate corresponding, analogous or similar elements, and in which:

FIG. 1 is a schematic representation of a treatment system according to embodiments of the invention;

FIG. 2 is a schematic view of a surface of an applicator in accordance with an embodiment of the invention;

FIG. 3 depicts energy fields according to embodiments of the invention;

FIG. 4A shows energy emission units according to embodiments of the invention; and

FIG. 4B shows energy emission units according to embodiments of the invention.

### DETAILED DESCRIPTION OF EMBODIMENTS OF THE INVENTION

In the following description, various embodiments of the invention will be described. For purposes of explanation, specific examples are set forth in order to provide a thorough understanding of at least one embodiment of the invention. However, it will also be apparent to one skilled in the art that other embodiments of the invention are not limited to the examples described herein. Furthermore, well-known features may be omitted or simplified in order not to obscure embodiments of the invention described herein.

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently.

The term “skin treatment” as used in this document may refer to treatment of indicated skin conditions. These may include cellulite reduction treatment, skin tightening, body contouring, skin toning, treating of pigment lesions, skin texture improving, scar reduction, wrinkle smoothing and treatments of other conditions. Skin treatment as described herein may include treating skin tissue in general or separate layers such as the epidermis, dermis and subcutaneous layers and some of the underlying layers, e.g., fat or muscle layers. For the sake of clarity and simplicity, skin layers treated according to embodiments of the invention are not specifically indicated hereinafter and it will be understood that any of the layers and/or tissues indicated above are contemplated hereinafter.

According to embodiments of the invention, treatment of skin tissue and layers may include applying electro-magnetic radiation or energy and ultrasound energy(US) to the selected or specific tissue. Energy applied as described may dissolve or otherwise affect adipose tissue or cellulite. Substance (e.g., cellulite) may be removed using negative pressure as further described.

Different RF and ultrasound (US) frequencies, amplitudes or other parametes may be used to treat different tissues, layers and depths. Accordingly, different tissues or layers may be treated without using large amounts of RF radiation or US energies thus avoiding undesirable effects to a skin and/or discomfort to a patient.

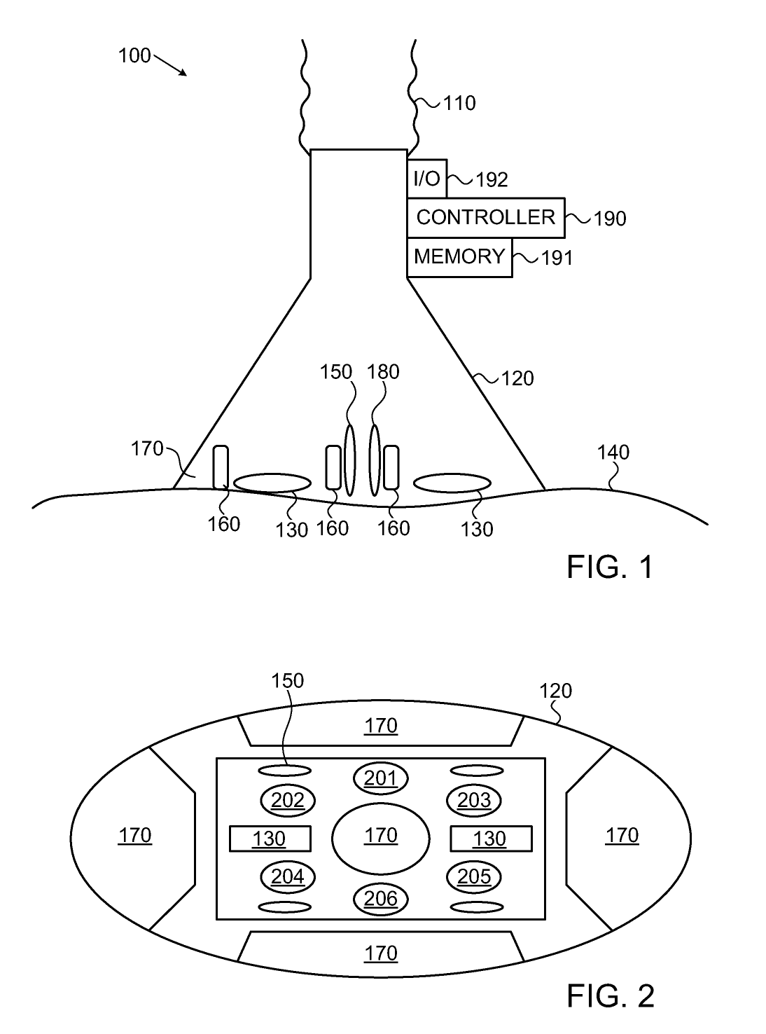

Reference is made to FIG. 1 that shows a schematic representation of a treatment system 100 according to embodiments of the invention. In some embodiments, skin treatment system 100 may be used to treat aesthetic conditions such as wrinkles, hyper pigmentation and for body shaping. Other uses and benefits may occur in other embodiments. In some embodiments, the skin treatment may involve treating subcutaneous tissue. As shown, system 100 may include a vacuum hose 110 that may be connected to a treatment applicator 120. A number of components may be installed in applicator 120. As shown, ultrasound transducers 130 may be flat or non-focused transducers that may be installed in applicator 120. As further shown, system 100 may include a cooling device 150, a plurality of radio frequency (RF) electrodes 160 and vacuum attachment zones 170. As shown, system 100 may include one or more sensors 180, a controller 190, a memory 191 and an Input/Output (I/O) system 192.

Treatment applicator 120 may be, or may include, a housing that may house a number of components as shown. Treatment applicator 120 may be placed on a skin surface of a patient in order to perform a treatment of a tissue using multiple energy types as described herein. A distal end of treatment applicator 120 may include a surface that may be held against a tissue surface (e.g., skin) and that may include a plate or treatment surface. For example, treatment applicator 120 may include a treatment surface having dimensions of approximately 10 mm by 15 cm. Other sizes, shapes and/or dimensions may be used. Treatment applicator 120 may include a treatment surface that may be placed in contact with, or proximate to, a skin surface to be treated. Energy emission components such as RF electrodes and (possibly flat and/or non-focused) ultrasound transducers may be installed in treatment applicator 120 such that they are at a desired distance or orientation with respect to treated tissues or with respect to skin surface 140.

For the sake of clarity and simplicity, some components of system 100 were omitted from FIG. 1. For example, controller 190 may be operatively connected to sensors 180, ultrasound transducers 130, RF electrodes 160 and/or cooling device 150, for example, using wired or wireless technologies. However, wires or other means of communications were omitted from FIG. 1. Similarly, embodiments of the invention may include large numbers of similar components (e.g., a large number of RF electrodes or ultrasound transducers). It will be understood that although a small number of components are shown in FIG. 1, any number of sensors 180, RF electrodes 160, ultrasound transducers 130 or other components shown in FIG. 1 may be included in a system without departing from the scope of the invention.

A number of vacuum attachment zones 170 may be included in system 100. For example, distributed on a surface of treatment applicator 120. A number of vacuum attachment zones 170 may be interspersed or placed among an array of energy emission components such as RF electrodes 160 and/or ultrasound transducers 130. Negative pressure (e.g., in the form of vacuum or suction) may be provided by a pump (not shown) that may be connected to vacuum hose 110 and may be carried to vacuum attachment zones 170 and/or a chamber or space within treatment applicator 120. Negative pressure or vacuum provided as described may enable tightening treatment applicator 120 to skin surface 140. Negative pressure or vacuum provided as described may enable removal of any substance from a space or region within treatment applicator 120. For example, liquids, particles, gas or debris released from skin surface 140 may be removed using suction produced by vacuum hose 110. In some embodiments, low or negative pressure may be sustained within treatment applicator 120 such that any substance released into a space within treatment applicator 120 may be removed, e.g., via vacuum hose 110.

RF electrodes 160 may generate an electro-magnetic field or radiation and may create and/or sustain a magnetic field on skin surface 140 or in any skin layer under skin surface 140, such as the epidermis, dermis and subcutaneous layers and in some of the underlying layer. Other treated tissues may be fat and/or muscle tissues. RF electrodes 160 may be any suitable RF electrodes configured to produce RF energy and may be provided with any density, or arrangement. For example, RF electrodes 160 may be approximately 100 to 1,000 microns in diameter and may be arranged around a predefined area, e.g., in a circle or other geometrical shape. RF electrodes 160 may emit RF energy according to various schemes, conditions or parameters. For example, controller 190 may cause RF electrodes 160 to emit RF energy at a frequency of 1 MHz, or within a frequency range of 0.2 MHz to 20 Mhz or RF electrodes 160 may be made to emit RF energy at a set of frequencies, e.g., 0.8, 1.7, 2.45 MHz.

Controller 190 may cause RF electrodes 160 to emit RF energy in pulses, e.g., pulses of 0.05-0.5 sec and total energy of 10 to 100 mj/pin and with a density of from 10 to 100 electrodes per square centimeter. Controller 190 may cause a first subset of RF electrodes 160 to operate according to a first configuration or parameter and cause a second subset of RF electrodes 160 to operate according to a second configuration, parameter or scheme. Accordingly, an amplitude, a polarity, a duration, an interval, a frequency, a period, a cycles, an energy level, or a pulse characteristic according to which RF electrodes emit RF energy may be set and/or controlled by controller 190.

Controller 190 may cause a set of RF electrodes and/or ultrasound transducers to generate RF and/or ultrasound energies at a fixed, constant or variable energy level. Any sequence of generation or emission of RF or ultrasound energies may be performed by a system. Where applicable, parameters such as amplitude, phase, wave lengths, polarity, frequency, cycle, duration, interval, cycle, aggrated energy level, or pulse characteristic may all be dynamically controlled by controller 190. Operation of RF electrodes 160 and ultrasound transducers 130 may be synchronized or correlated by controller 190. For example, energy levels and types in a specific skin layer may be maintained by causing RF electrodes 160 sustain a first portion of the energy in a region and causing ultrasound transducers 130 to sustain a second portion of the energy in the same region. Other methods and/or systems for operating RF electrodes 130 may be as described in U.S. patent publication No. 2010/0087899 of U.S. patent application Ser. No. 12/245,374 filed Oct. 3, 2008 by the an inventor of the present application and entitled “SKIN TREATMENT USING VARIABLE RF” which is hereby incorporated by reference.

Controller 190 may activate or deactivate any component according to any sequence, rule or criteria. For example, two RF electrodes to emit RF energy may be selected based on the distance between the two RF electrodes, e.g., in order to maximize RF energy in a specific tissue.

Cooling device 150 may be any suitable cooling device or system, e.g., a fan, a thermoelectric cooling device or a system designed to cause a flow of cold air such that any region, space or component of system 100 are cooled. Cooling device 150 may be controlled by controller 190. For example, based on information provided by one of sensors 180 (e.g., a temperature sensor) controller 190 may activate cooling device 150 when temperature inside treatment applicator 120, or temperature on skin surface 140 reaches a threshold. Sensors 180 may include any suitable sensors. For example, pressure, temperature, energy levels may all be monitored or sensed by sensors 180. Levels or other attributes of an RF field or ultrasound waves may be monitored and/or sensed by sensors 180. Any parameter sensed or monitored by one or more of sensors 180 may be provided to controller 190. Accordingly, operation of system 100 may be dynamically controlled and/or adapted based on dynamic parameters such as temperature, pressure, heat, energy levels etc. Sensors 180 may sense or measure parameters such as an electrical resistance or impedance of a skin or other tissue. In other embodiments or configuration, a pigmentation or other attributes of a skin may be measured, sensed or monitored. In other embodiments, sensors 180 may include a component for emitting infra red (IR) light and an IR sensor. Accordingly, absorption and/or reflection or emission of IR may be measured. Various parameters may be determined based on reflected or absorbed IR light, for example, a temperature or blood flow may be measured.

As described herein, an operation of a system may be according to sensed, measured or monitored parameters. For example, a first energy level (e.g., RF or ultrasound energy) may be generated for a first patient and a second energy may be generated for a second patient based on a measured or sensed skin or layer attribute.

As described herein, embodiments of the invention may cause cells to vibrate in order to increase absorption of RF energy. Although the discussion herein mainly refers to vibrating cells using ultrasound transducers, other means may be contemplated. For example, mechanical means may be used in order to cause vibration of cells. Accordingly, where applicable, ultrasound transducers discussed herein may be replaced by other means of generating a vibration. Ultrasound transducers 130 may be any device configured to produce acoustic energy at ultrasound wavelengths. For example, ultrasound transducers 130 may be flat and/or non-focused transducers. Operation of ultrasound transducers 130 may be controlled by controller 190. For example, based on parameters sensed by sensors 180, controller 190 may alter an operational parameter of ultrasound transducers 130. Accordingly, a wavelength, amplitude, pulse or other aspects of ultrasound energy and/or sound waves produced by ultrasound transducers 130 may be automatically set or may be dynamically changed. For example, based on input from a heat sensing device (e.g., included in sensors 180), an amplitude or duration of ultrasound waves produced by transducers 130 (or RF energy produced by RF electrodes 160) may be automatically decreased, e.g., if a temperature of skin surface 140 exceeds a limit or threshold.

Controller 190 may be a central processing unit processor (CPU), a chip or any suitable computing or computational device. Memory 191 may be or may include, for example, a Random Access Memory (RAM), a read only memory (ROM), a Dynamic RAM (DRAM), a Synchronous DRAM (SD-RAM), a double data rate (DDR) memory chip, a Flash memory or other suitable memory units or storage units. Memory 191 may be or may include a plurality of, possibly different memory units. Memory 191 may store any information, data or parameters related to an operation of system 100. For example, configuration parameters that may define an energy level emitted by RF electrodes 160 or ultrasound transducers 130 may be stored in memory 191.

Input and output (I/O) system 192 may include user interface buttons and/or screens. Foe example, I/O system 192 may include a mouse, a keyboard, a touch screen or pad or any suitable input device and any components required to connect user interface devices to controller 190. It will be recognized that any suitable number of input devices may be operatively connected to controller 190. I/O system 192 may include one or more displays, speakers and/or any other suitable output devices. It will be recognized that any suitable number of output devices may be operatively connected to controller 190. Any applicable I/O devices or systems may be connected to controller 190 as shown by I/O system 192. For example, a wired or wireless network interface card (NIC), a modem, printer or a universal serial bus (USB) device or external hard drive may be included I/O system 192. For example, I/O system 192 may include hardware, software and/or firmware that may enable receiving operational parameters from a remote device (e.g., a smartphone). Parameters received may be stored in memory 191 and may be used, e.g., by controller 190, in order to control an operation of system 100. Although shown coupled to treatment applicator 120, controller 190, memory 191 and/or I/O system 192 may be placed remotely.

For example, using I/O system 192, controller 190 may communicate with a remote computing device. The remote computing device may be equipped with human interface systems (e.g., a display, mouse and/or keyboard). In other embodiments, controller 190 and/or memory 191 may be included in a remote device and may communicate with components of system 100 via I/O system 192. Accordingly, system 100 may be remotely operated, monitored and/or controlled. System 100 may include additional elements not shown in FIG. 1. For example, system 100 may include (or be connected to) a power or energy source (e.g., batteries or means for connecting to a power supply). System 100 may include a container, receptacle or reservoir for collecting material released from skin surface 140.

Embodiments of the invention (e.g., system 100) may enable improving skin appearance using noninvasive means and low levels of energy. Accordingly, embodiments of the invention may enable improving skin appearance using a mild treatment with minimal damage to skin and minimal patient discomfort and/or side effects. As described herein, several modalities of treatment may be combined and operated at same treated area, either simultaneously or at a predefined sequence.

In an embodiment, a treated area may be subjected to vacuum negative pressure combined with ultrasound energy and RF energy. RF energy causes a thermal effect which increases cell walls permeability. In addition, RF energy enhances circulation in energized tissues. As discovered, tissues and cells response to RF energy is higher when they are vibrated close to their resonance frequency. Thus the RF energy absorption is better when cells are vibrating due to mechanical or ultrasound enhanced vibrations while RF energy is applied. Accordingly and as described herein, a treated tissue may be subjected ultrasound energy that may cause cells to vibrate and to RF energy that may cause a thermal effect. As described, a treated area may also be cooled to relive pain and avoid edema, erythema and redness that may be caused by heating the skin surface.

Vacuum negative pressure is known to cause improvement of skin appearance by enhancing local blood flow, collagen formation and other effects. As described herein, vacuum or negative pressure may be applied by a system according to embodiments of the invention such that an apparatus is made to remain stationary on a skin of a patient. Vacuum or negative pressure may be applied by a system in order to increase blood flow or affect collagen formation.

A system according to embodiments of the invention may include an ultrasound transducer configured to produce sound waves at a surface of the skin and a plurality of RF electrodes configured to emit one or more RF signals. The ultrasound transducer may generate ultrasound waves that may sustain energy at a predefined or selected tissue. For example, controller 190 may control the ultrasound transducer to generate ultrasound waves such that a substantial amount of the energy of the ultrasound waves is confined to a specific depth, or a specific tissue of the skin For example, by controlling the frequency or amplitude of the sound waves generated by an ultrasound transducer, controller 190 may maximize the energy absorbed in a specific depth, tissue or layer.

A system according to embodiments of the invention may include at least two ultrasound transducers configured to produce sound waves at a surface of the skin and a plurality of RF electrodes configured to emit one or more RF signals. Sound waves transducers included in a system may be configured to emit sound waves such that a constructive interference of the sound waves sustains a predefined level of energy at a specific tissue below the surface of the skin.

For example, the distance between two of ultrasound transducers 130 may be such that a constructive interference of sound waves generated by the two transducers occurs at a specific depth under skin surface 140. Additionally or alternatively, characteristics of sound waves generated by one or more transducers included in ultrasound transducers 130 may be controlled (e.g., by controller 190) such that an energy level at a selected depth or layer is maximized. For example, controller 190 may control the wave length, amplitude or frequency of sound waves generated by two or more transducers such that the resulting or combined energy at a specific skin layer is within a predefined range.

According to embodiments of the invention, a first and second ultrasound transducers (e.g., included in ultrasound transducers 130) may produce sound waves according to a first and second parameters (e.g., a wave lengths, a phase, polarity and/or amplitude) in order to sustain a respective first and second predefined level of energies at a respective first and second layers of a skin. For example, two or more non-focused ultrasound transducers may be caused to produce two or more sound waves such that the combined energies of the two or more sound waves peak at two or more regions, layers or depths. As known in the art, a wave of any form may be characterized by a (possibly large) number of waves. For example, using Fourier analysis and/or transform, any function or wave in space may be produced, e.g., using a series of sines and cosines and associated amplitudes that, when superimposed, will reproduce the desired function or wave. Accordingly, two or more of ultrasound transducers 130 may be operated to produce a first and second sequences of sound waves such that an amplitude of a combined sound wave will peak in a first and second regions, depths or volumes. For example, two separate tissues or layers of a skin may be heated or energized to respective two different levels of heat or energy.

In an embodiment, two or more ultrasound transducers may be respectively positioned such that they produce a constructive interference of sound waves at a specific layer of the skin For example, a number of ultrasound transducers may be rotated, tilted or otherwise respectively positioned such that an interference of sound waves produced by the transducers causes cells at a specific skin layers to vibrate. For example, by respectfully positioning two or more transducers such that an energy level at a specific skin layer is maximized or otherwise controlled, cells in the specific skin layer may be caused to vibrate at their resonance frequency. Otherwise described, by placing a first transducer at a specific distance from a second transducer, and/or at a specific respective orientation, the skin layer where a constructive interference of sound waves generated by the first and second transducers will occur may be selected.

Embodiments of the invention enable treating one or more tissues, e.g., tissues or layers of a human skin A number of tissues may be selected and treated. In an embodiment, a plurality of tissues may be treated simultaneously by a single device, in other embodiments, the same system or device may treat a number of tissues sequentially. For example, system 100 may be configured to generate and sustain a first energy level in a first skin layer during a first period of time and generate and sustain a second energy level in a second skin layer during a second period of time. In other embodiments, system 100 may be configured to generate and sustain a first and second energy levels in a first and second skin layers simultaneously.

In particular, embodiments of the invention may generate, sustain and/or maintain an energy level in a selected tissue, layer or depth using ultrasound and RF energy generators. Different depths or layers may be treated or energized using a single frequency and/or multiple frequencies of RF energy generated by RF electrodes. Various attributes, configurations or parameters related to RF energy applied by a system may be set, configured and/or controlled. For example, parameters such as a frequency, an amplitude, a phase and/or a polarity of RF energy may be controlled, set and/or altered by a user and/or by a system. As further described herein, selected subsets of RF electrodes may be caused to operate according to selected sets of parameters. Similarly, various attributes, configurations or parameters related to ultrasound energy applied by a system may be set, configured and/or controlled. For example, parameters such as a frequency, an amplitude, and/or a phase of ultrasound energy may be controlled, set and/or altered by a user and/or by a system.

Reference is now made to FIG. 2 that shows a schematic view of a surface of an applicator in accordance with an embodiment of the invention. As shown, RF electrodes 201-206 may be arranged in a circle around a treatment area. Other arrangement may be possible, for example, electrodes may be arranged according to a density parameter (e.g., a specific number of electrodes per square inch. Electrodes may be added or removed based on a configuration or they may be dynamically and/or automatically made active or inactive based on a configuration parameter or based on conditions. For example, based on a heat level or other measured parameters electrodes may be activated or deactivated by controller 190.

In an embodiment or scenario, two or more at least two of the RF electrodes 201-206 may be caused to produce a bipolar RF signal according to a predefined sequence. For example, a selected RF electrode, e.g., electrode 201, and a first subset of RF electrodes, e.g., electrodes 202 and 203, are used to induce a first electro-magnetic field in a first layer of the skin Subsequently or simultaneously, the selected RF electrode (electrode 201 in this example) and a second subset of RF electrodes (e.g., electrodes 204 and 205) may be used to induce a second electro-magnetic field in a second layer of the skin Other sequences and polarities may be possible.

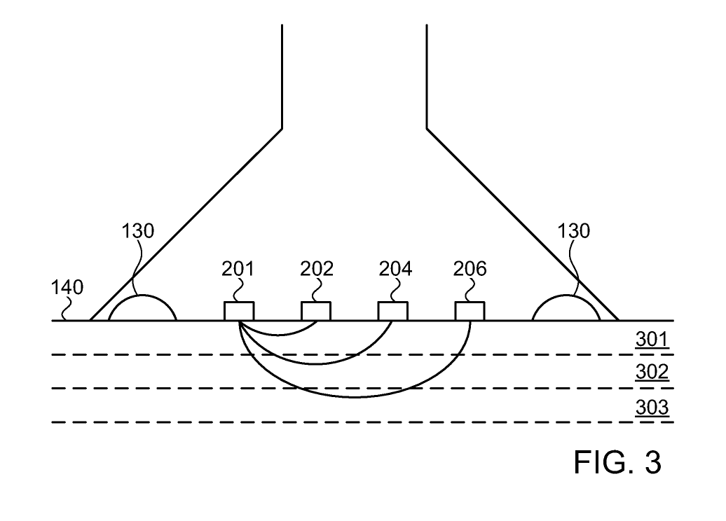

Reference is additionally made to FIG. 3 that schematically shows energy fields according to embodiments of the invention. As shown, a number of layers 301, 302 and 303 may be treated separately. In some embodiments, layers 301-303 may be treated simultaneously. Although not shown in FIG. 3, ultrasound energy produced by transducers 130 may be concentrated in any one of layers 301-303, e.g., by tuning parameters governing operation of transducers 130 such that a major portion of a combination of energies generated by transducers 130 is present in a specific one of layers 301-303. For example and as discussed herein, the distance between transducers 130 shown in FIG. 3 and/or a frequency of generated sound waves may be set such that a constructive interference of ultrasound waves generated by transducers 130 occurs within layer 302. Controller 190 may control RF electrodes and ultrasound transducers such that RF energy and ultrasound energy are concentrated in one of layers 301-303.

Referring to FIG. 3, RF frequency may be set to a range of 0.1-10 MHz. RF electrode 201 may act as a positive (+) pole and RF electrodes 202 (and possibly RF electrode 203, not shown in FIG. 3) may act as a negative (−) pole to induce a magnetic field with a specific polarity in layer 301 as shown. Alternatively or additionally, RF electrode 201 may act as a negative (−) pole and RF electrodes 204 (and possibly RF electrode 205, not shown in FIG. 3) may act as a positive (+) pole to induce an electrical field with a specific polarity in layer 302 as shown. Similarly, an electrical field in layer 303 as shown may be produced using RF electrode 201 and RF electrode 206. Accordingly, by setting the distance between electrodes and the frequency of the generated RF energy, electro-magnetic fields and energies may be provided at specific layers. The distance between operative electrodes or between electrodes that cause an energy field may be dynamically set. For example, controller 190 may select a first set of electrodes at a first distance from each other to operate during a first period of time and a second set of electrodes at a second distance from each other to operate during a second period time. In other cases, the set of electrodes (and, consequently, their respective distances from one another) may be selected for an entire treatment. With respect to a polarity of an energy field, in addition or instead of a bipolar field, a multipolar field may be generated. For example, by continuously and/or dynamically switching roles assumed by electrodes, a multipolar field may be generated. For example, controller 190 may cause a set of electrodes to continuously alternate roles, e.g., act as a positive (+) pole for a (possibly small) duration of time and as a negative (−) pole for a subsequent period of time. Accordingly, using a number of electrodes, any polarity may be achieved, e.g., a multipolar, time dependent field may be generated.

Moreover, embodiments of the invention may generate different electro-magnetic fields and energies using a single RF frequency, e.g., by selecting a frequency range of 0.1-10 MHz and selectively activating different sets of electrodes as described herein. In some embodiments, a number of frequencies may be used, e.g., in order to create different electro-magnetic fields at respective different depths using a fixed set of electrodes.

By controlling a location and/or orientation of energy generation units, energy may be confined to a specific layer or volume to be treated. By controlling energy generation parameters such as amplitude, frequency, polarity and/phase, specific energy levels of respective specific energy types may be maintained in specific layers or volumes to be treated. Controller 190 may be configured to control any applicable aspect related to energy generation units. Controller 190 may receive input from a user (e.g., via user interface as described herein) and control RF electrodes 160 and ultrasound transducers 130 according to input from a user. Controller 190 may set operational or other parameters automatically, e.g., based on a duration of a treatment or based on input from one of sensors 180. For example, a predefined sequence of operation including treating a first skin layer using a first energy level and then treating a second skin layer using a second energy level may be stored in memory 191 and controller 190 may use such stored sequence to cause system 100 to apply a treatment according to the sequence.

A treatment or aspects of a treatment may be determined automatically. For example, using ultrasound transducers 130 and an ultrasound sensing device, presence, location, density or other attributes of a skin layer may be determined, e.g., by measuring absorption and/or reflection of light or ultrasound waves. For example, controller 190 may use ultrasound transducers 130 and one of sensors 180 to measure ultrasound energy reflection or absorption of tissues and thus determine attributes of tissues to be treated. Based on determined attributes of tissues (e.g., location, density and the like), controller 190 may automatically set operational parameters as described herein such that a suitable energy levels and fields are maintained in appropriate tissues. In some embodiments, controller 190 may relocate or position ultrasound transducers 130 and/or RF electrodes 160 in order to achieve a desirable configuration, e.g., by controlling servomotor capable of moving or rotating ultrasound transducers 130 and/or RF electrodes 160. Any combination of RF and US frequencies may be used. For example, US energy in a range of 1 MHz to 3 MHz may be combined with any of the RF frequencies discussed herein. Using flat, non-focused US transducers, frequencies higher than otherwise possible may be enabled. For example, frequencies that may need to be avoided when focused US transducers are used may safely be used in systems according to embodiments of the invention.

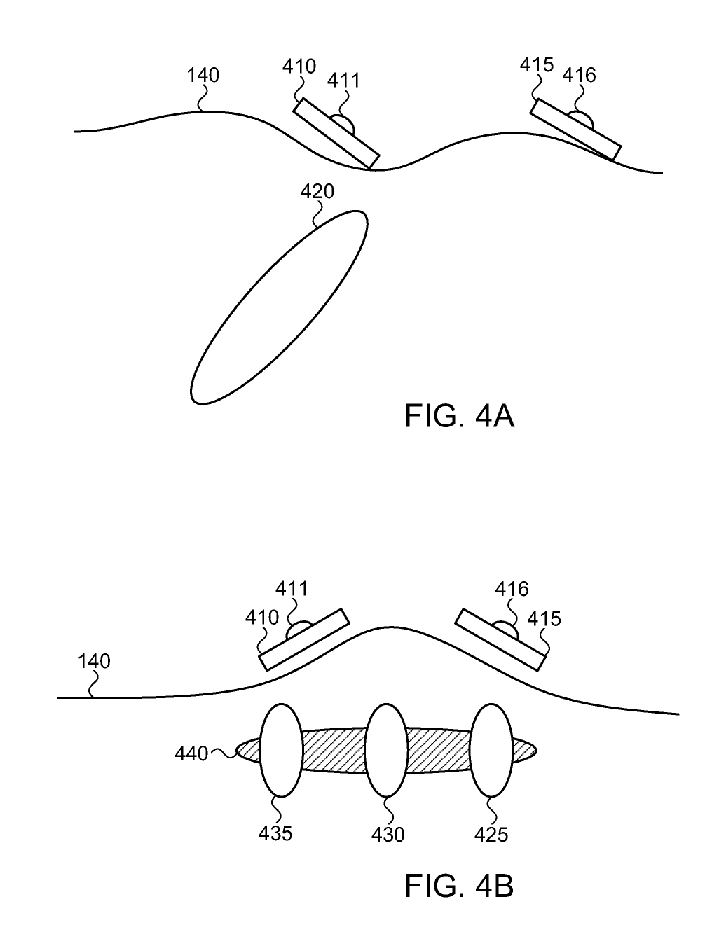

A direction in which energy is emitted or propagated may be dynamically or otherwise controlled. For example, RF electrodes 160 and ultrasound transducers 130 may be mounted on treatment applicator 120 such that their location, position and/or orientation may be altered by a user, e.g., by suitable mounting components. In other embodiments, controller 190 may change a location, position or orientation of RF electrodes 160 and/or ultrasound transducers 130. Reference is now made to FIGS. 4A and 4B that show energy emission units according to embodiments of the invention. It will be understood that although, for the sake of simplicity, ultrasound transducers are mainly discussed with respect to FIGS. 4A and 4B, the discussion herein applies to RF electrodes. Any methods of directing energy as discussed herein with respect to ultrasound transducers may be implemented for RF electrodes or other forms of energy. As shown in FIG. 4A, possibly causing a slight and temporary deformation of skin layer 140 as shown, ultrasound transducers 410 and 415 may be tilted in the same direction by respective servomotors 411 and 416. For example, controller 190 may control servomotors 411 and 416 and may, accordingly, position ultrasound transducers 410 and 415 as shown. For example, a region as shown by 420 may be energized by a combination of sound waves generated by ultrasound transducers 410 and 415 when positioned as shown. Any respective orientation of ultrasound transducers 410 and 415 may be assumed, accordingly, any specific region or layer may be selected for treatment. By properly positioning ultrasound transducers 410 and 415 a number of discrete, different or separate regions may be energized or a specific layer may be treated.

For example and as shown in FIG. 4B, an interference of waves generated by ultrasound transducers 410 and 415 may heat or otherwise energize regions 425, 430 and 435. For example, wave lengths may be controlled such that an interference of waves generated by ultrasound transducers 410 and 415 is constructive in regions 425, 430 and 435. In other cases, a layer as shown by 440 may be treated. For example, ultrasound transducers 410 and 415 may be continuously moved and/or tilted such that a specific region or layer are treated.

Ultrasound transducers 410 and 415 may be positioned at a specific respective or relative position or angle. For example, the angle between ultrasound transducers 410 and 415 as in FIG. 4B may be set to 70° (seventy degrees). The angle (or other positioning parameters) may be dynamically and/or continuously controlled and changed. For example, using servomotor as discussed herein, ultrasound transducers 410 and 415 may be caused to dynamically assume a range of relative angles or positions.

While the invention has been described with respect to a limited number of embodiments, it will be appreciated by persons skilled in the art that the present invention is not limited by what has been particularly shown and described herein. Rather the scope of the present invention includes both combinations and sub-combinations of the various features described herein, as well as variations and modifications which would occur to persons skilled in the art upon reading the specification and which are not in the prior art. While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

What is claimed is:

1. A system for treating skin, comprising:
At least two flat transducers configured to produce sound waves at surface and inner layer of the skin;
a plurality of radio frequency (RF) electrodes configured to emit a plurality of RF signals; and
a control unit to control the transducers and the electrodes, wherein an interference of the sound waves sustains a predefined level of energy at a specific tissue below the surface of the skin

2. The system of claim 1, wherein the control unit is configured to cause the transducers to produce sound waves according to a first and second parameters in order to sustain a respective first and second predefined level of energies at a respective first and second layers of the skin

3. The system of claim 1, wherein the at least two transducers are respectively positioned to produce a constructive interference of sound waves at a specific layer of the skin

4. The system of claim 1, wherein at least two of the RF electrodes to produce a bipolar RF signal according to a predefined sequence.

5. The system of claim 1, wherein a subset of the RF electrodes is to produce a multipolar RF signal.

6. The system of claim 1, wherein a selected RF electrode and a first subset of RF electrodes are used to induce a first electro-magnetic field in a first layer of the skin and the selected RF electrode and a second subset of RF electrodes are used to induce a second electro-magnetic field in a second layer of the skin

7. The system of claim 1, wherein a first and second RF electrodes are selected based on a distance between the first and second RF electrodes.

8. The system of claim 1, wherein the sound waves are generated such that cells included in a specific layer of the skin vibrate at a resonance frequency of the cells.

9. The system of claim 1, comprising a negative pressure outlet to apply a negative pressure to an area of the skin and a receptacle to collect materials released from the skin

10. The system of claim 1, comprising a cooling unit to cool an area of the skin

11. The system of claim 1, comprising a sensor and wherein the at least one control unit is to control the RF electrodes and the transducers according to an input from the sensor.

12. The system of claim 1, wherein the control unit is to control the RF electrodes and the transducers according to an input from a user.

13. The system of claim 1, wherein the plurality of RF electrodes are arranged substantially around the area of the skin

14. The system of claim 1, wherein sound waves are ultrasound waves.

15. The system of claim 1, comprising means for producing negative pressure to remove substance from a treated area.

16. A method of treating skin, comprising:
providing at least two flat transducers configured to produce sound waves at surface and inner layer of the skin;
providing a plurality of radio frequency (RF) electrodes configured to emit a plurality of RF signals; and
causing the transducers to generate sound waves such that an interference of the sound waves sustains a predefined level of energy at a specific tissue below the surface of the skin

17. The method of claim 16, wherein the control unit is configured to cause the transducers to produce sound waves according to a first and second parameters in order to sustain a respective first and second predefined level of energies at a respective first and second layers of the skin

18. The method of claim 16, wherein the at least two transducers are respectively positioned to produce a constructive interference of sound waves at a specific layer of the skin.

19. The method of claim 16, wherein at least two of the RF electrodes to produce a bipolar RF signal according to a predefined sequence.

20. The method of claim 16, wherein a subset of the RF electrodes is to produce a multipolar RF signal.
